(19) United States
(12) Patent Application Publication
(10) Pub. No.: US 2013/0172736 A1
(43) Pub. Date: Jul. 4, 2013

(54) DEVICES AND METHODS FOR THE DIAGNOSIS AND TREATMENT OF SACRO-ILIAC JOINT DISEASE

(71) Applicant: Samy ABDOU, San Diego, CA (US)

(72) Inventor: Samy ABDOU, San Diego, CA (US)

(73) Assignee: Samy ABDOU, San Diego, CA (US)

(21) Appl. No.: 13/733,832

(22) Filed: Jan. 3, 2013

Related U.S. Application Data
(60) Provisional application No. 61/631,356, filed on Jan. 3, 2012.

(51) Int. Cl. A61B 17/70; A61B 6/03; A61M 5/00

(52) U.S. Cl. CPC A61B 17/70; A61M 5/00; A61B 6/03
USPC 600/425; 606/279; 604/506

(57) ABSTRACT

Methods and apparatus for diagnostic and/or therapeutic manipulation of the sacro-iliac joint (SI joint). Trajectories for corridors used to manipulate the target SI joint are also disclosed. In one embodiment, the method comprises palpitating the inferior end of the SI joint, confirming via an imaging technique, and injecting a material into the joint. The injected material (such as a bone forming material) is deposited from the needle or through an implanted catheter. The SI joint may be also immobilized while the fusion mass forms via the use of at least one fixation member. In one embodiment, the fixation member comprises an external member and two internal members which threadedly engage each other. The fixation member is configured to expand and comprises bone growth promoting feature. The expanded member fixates the ilium to the Sacrum on the side of the target SI joint, thus the SI joint is immobilized and fused.

### PRIORITY

The present disclosure claims priority to commonly owned U.S. Provisional Patent Application Ser. No. 61/631,356 filed on Jan. 3, 2012 and entitled “DEVICES AND METHODS FOR THE DIAGNOSIS AND TREATMENT OF SACRO-ILIAC JOINT DISEASE”, which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

1. Technological Field

The disclosure relates generally to the field of spinal disease diagnosis and treatment. In one exemplary aspect, the disclosure relates to devices and methods for sacro-iliac joint (SI joint) injection, fusion and/or fixation.

2. Description of Related Technology

Pain from degenerative spine disease is a major health problem in the industrialized world and the surgical treatment of spinal pathology is an evolving discipline. The traditional surgical treatment of a degenerating and painful inter-vertebral disc has been the complete immobilization and bony fusion of the involved spinal segment. An extensive array of surgical techniques and implantable devices have been formulated to accomplish this goal.

The growing experience with spinal fusion has provided considerable data on the efficacy of using vertebral immobilization to alleviate low back pain. Despite the high fusion rates that are achievable in current surgical practice, about 20-30% of patients who undergo successful lumber fusion continue to experience significant post-operative low back pain. This realization has fueled the search for additional sources of low back pain. To that end, the sacro-iliac joint (SI joint) has been implicated as a potential source of significant low back pain. The SI joint functions to transmit the load borne by the spine onto the pelvis and, while an anatomic component of the pelvis, it can be functionally considered to the inferior-most joint of the spinal column.

In current practice, pain relief after injection of an anesthetic substance into an SI joint suspected of being the source of the patient's low back is used a confirmation of the painful nature of the injected joint. When the painful SI joint becomes a chronic source of pain, the joint may be immobilized with a bone screw and/or fused with a bone segment in hopes of ameliorating the ongoing symptoms.

In this application, a novel device and method for SI joint injection, fusion and/or fixation are disclosed. The features and advantages of the present invention will become apparent from the following description and accompanying drawings.

### SUMMARY

The foregoing needs are addressed herein by providing, inter alia, methods and apparatus for the diagnosis and treatment of Sacro-iliac joint disease. Specifically devices and methods are disclosed for the diagnostic and/or therapeutic manipulation of a target SI joint that is positioned between an adjacent Sacrum bone and ilium bone.

Trajectories for the corridors used to manipulate the target SI joint are disclosed. In a first trajectory, the region of the inferior end of the target SI joint is accessed. A corridor is then formed in a cephalad direction towards the superior end of the target SI joint, the corridor is substantially parallel to the longitudinal axis of the target SI joint and is positioned at least partially within the target SI joint or within the posterior sacroiliac ligament immediately posterior to the target SI joint.

In a second trajectory, the region of the inferior end of the target SI joint is accessed. A corridor is formed in a cephalad direction towards the ipsilateral sacral ala, the corridor enters the ilium bone immediately lateral to the target SI joint, extends medially across a segment of the target SI joint (or a segment of the posterior sacro-iliac ligament positioned posterior to the target SI joint), and extends into a lateral aspect of the ipsilateral sacrum.

In a third trajectory, the region of the inferior end of the target SI joint is accessed. A corridor is formed in a cephalad direction towards superior end of the target SI joint. The corridor enters the posterior surface of the sacrum bone medial to the target SI joint, extends laterally across at least a segment of the target SI joint (or a segment of posterior sacro-iliac ligament positioned posterior to the target SI joint), and extends into a medial aspect of the ipsilateral ilium;

In a preferred method of fusion of the SI joint, a cavity is created that spans at least a segment of the target SI joint (or a segment of posterior sacro-iliac ligament positioned posterior to the target SI joint). At least a portion of the cavity extends in the direction of the first trajectory. At least one bone fastener is positioned across the target SI joint in order to immobilize it, and is positioned in a trajectory that forms an angle of 65 to 115 degrees relative to the longitudinal axis of the target SI joint.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIGS. 1A and 1B are perspective views of the bony components of an exemplary pelvis.

FIG. 2A is an anterior oblique view of an exemplary pelvis.

FIG. 2B is a posterior view of an exemplary pelvis.

FIG. 3 is a cross-sectional view of an exemplary sacro-iliac (SI) joint.

FIG. 4 is a posterior view of an exemplary SI joint for injection of an anesthetic agent therein.

FIG. 5A is a cross-sectional view of an exemplary SI joint.

FIG. 5B is a cross-sectional view of the exemplary SI joint of FIG. 5A using a bone screw for fixation thereof.

FIG. 6 is a posterior view of an exemplary pelvis illustrating an exemplary trajectory for one or more bone screws for fixation of the SI joint.

FIG. 7A is a posterior view of an upper aspect of the right lower extremity illustrating approximate locations of the SI joint and an exemplary trajectory.

FIG. 7B is a transverse X-ray computed tomography (CT) section of the Gluteus Maximus muscle illustrating an exemplary trajectory for accessing the SI joint.

FIGS. 8A-8C are magnified views of the SI joint illustrating exemplary trajectories A-C for accessing the SI joint.

FIG. 9 is a cross-sectional view of a first exemplary trajectory used for implanting bone forming material.

FIG. 10 is a cross-sectional view of an exemplary SI joint illustrating implantation along the trajectory of FIG. 9.

FIG. 11 is a posterior view of a second exemplary trajectory for the insertion of a fixation member.

FIG. 12A is a posterior view of a third exemplary trajectory for the insertion of a fixation member.

FIG. 12B is a lateral view of the third exemplary trajectory for the insertion of a fixation member.

FIG. 13A is a perspective view of an exemplary fixation member for use with the exemplary trajectories of FIGS. 8A-8C.

FIG. 13B are side and top views of an exemplary fixation member for use with the exemplary trajectories of FIGS. 8A-8C.

FIG. 14A is cross-sectional view of an exemplary fixation member for use with the exemplary trajectories of FIGS. 8A-8C.

FIG. 14B is an exploded view of an exemplary fixation member for use with the exemplary trajectories of FIGS. 8A-8C.

All Figures© Copyright 2012-2013. All rights reserved.

### DETAILED DESCRIPTION

Reference is now made to the drawings wherein like numerals refer to like parts throughout.

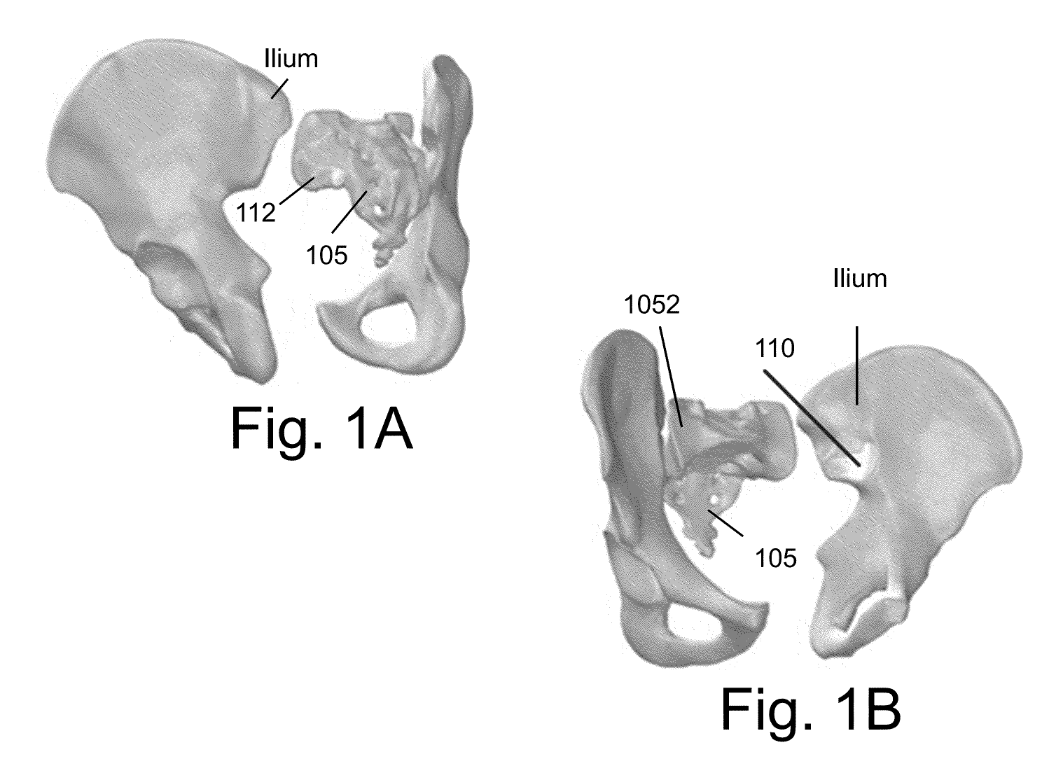

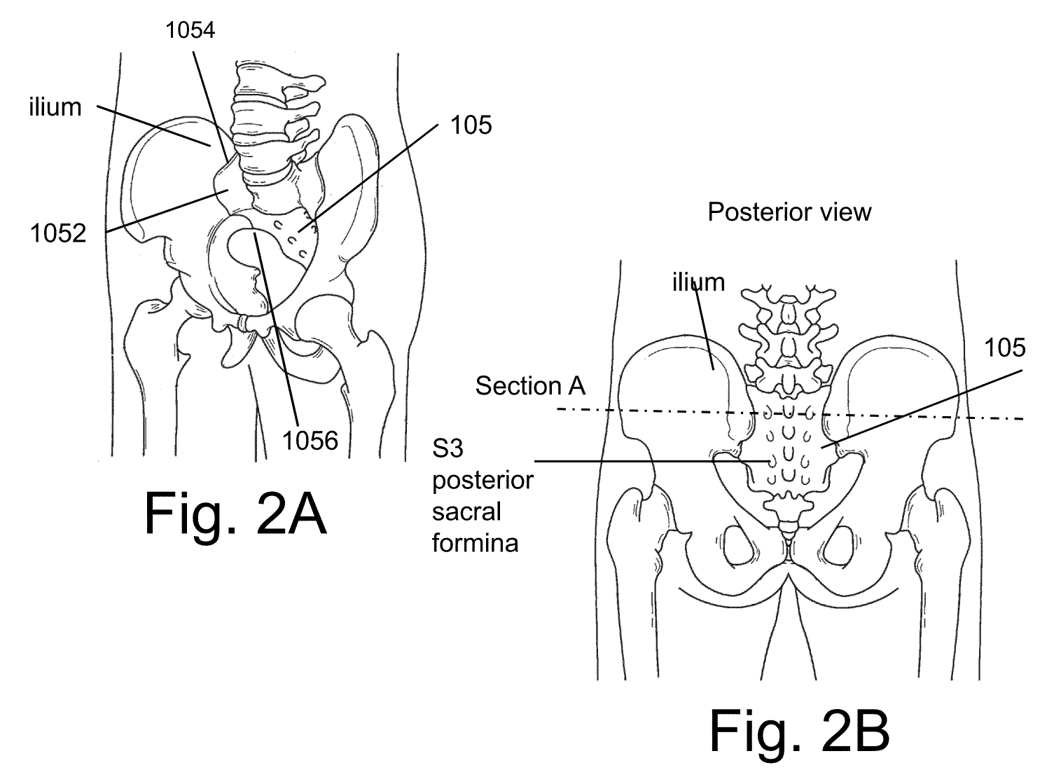

FIG. 1 illustrates the bony components of the pelvis. The sacrum 105 articulates with the ilium at the sacro-iliac joint (SI joint). The sacral surface of the sacro-iliac joint is labeled 112 whereas the ilium surface of the sacro-iliac joint is labeled 110. The superior aspect of the sacral ala is labeled 1052. Illustrations of the pelvis are shown in FIGS. 2A and 2B, wherein an anterior oblique view is shown in FIG. 2A and a posterior view is shown in FIG. 2B. The sacro-iliac joint extends from a superior end 1054 to an inferior end 1056 (with the subject standing erect and upright). Note that the ala of the sacrum 1052 is at the superior aspect of the sacrum and in proximity to end 1054. Further, the inferior end 1056 of SI joint is at the approximate level of the S3 pedicle and S3 nerve root in the horizontal plane.

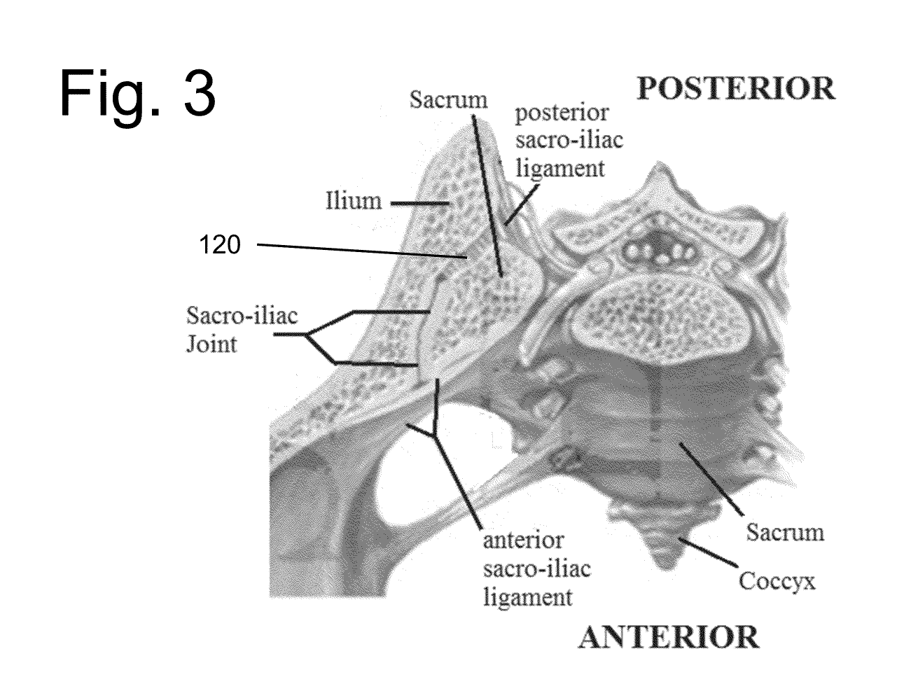

A cross-section of the sacro-iliac joint is shown in FIG. 3. The illustrated cross-section is at approximate level of “section A” of FIG. 2B—which is in proximity to the SI neural foramina and nerve roots. As can be seen, a space 120 rests posterior to the sacro-iliac joint and contains fibers of the posterior sacro-iliac joint.

The preceding illustrations and definitions of anatomical structures are known to those of ordinary skill in the art. The anatomy of the pelvis in general, and the sacroiliac joint in particular, are described in more detail in Atlas of Human Anatomy, by Frank Netter, third edition, Icon Learning Systems, Teterboro, N.J. The text is hereby incorporated by reference in its entirety.

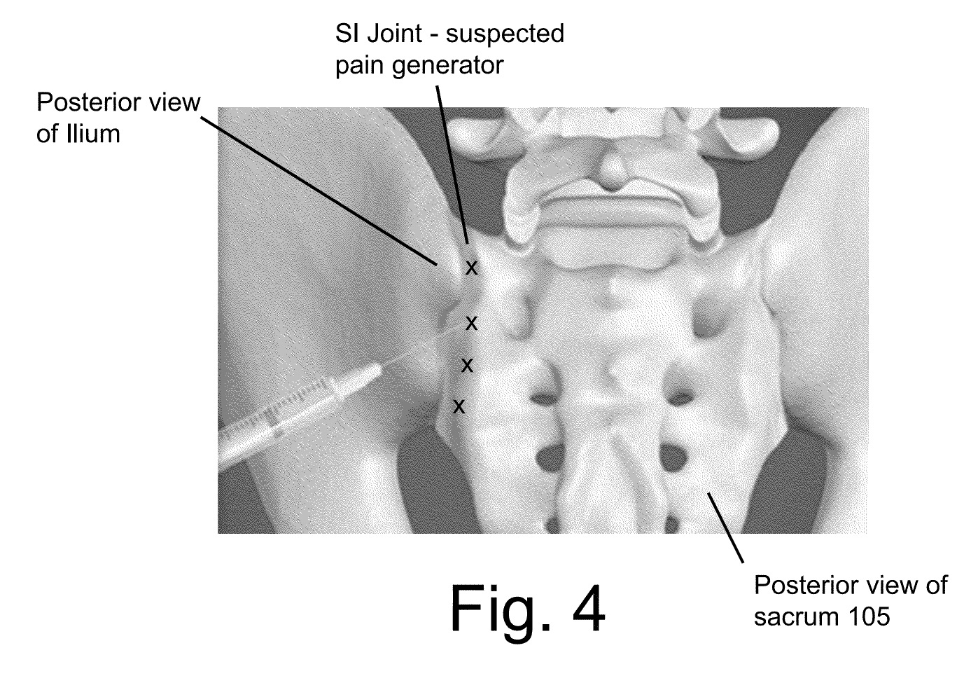

In current practice, an SI joint that is suspected of being the source of low back pain is injected with an anesthetic agent. Relief of the patient's pain symptoms, even when temporary, is used to confirm that the injected joint is in fact the source of pain (often termed the “pain generator”). FIG. 4 shows a posterior view of the SI joint and illustrates this technique. A syringe and needle are used to repeatedly inject an SI joint at multiple points “X”. In this technique, the joint is injected substantially in the axial plane. The needle is withdrawn from the patient's body cavity and reinserted through the skin at additional superior or inferior points. That is, in current practice, provocative injection to localize the SI joint pain generator is performed by repeated injections. Usually, the needle is substantially positioned in an axial plane relative to the joint to be injected. Note that the joint anatomically extends from a superior end to an inferior end.

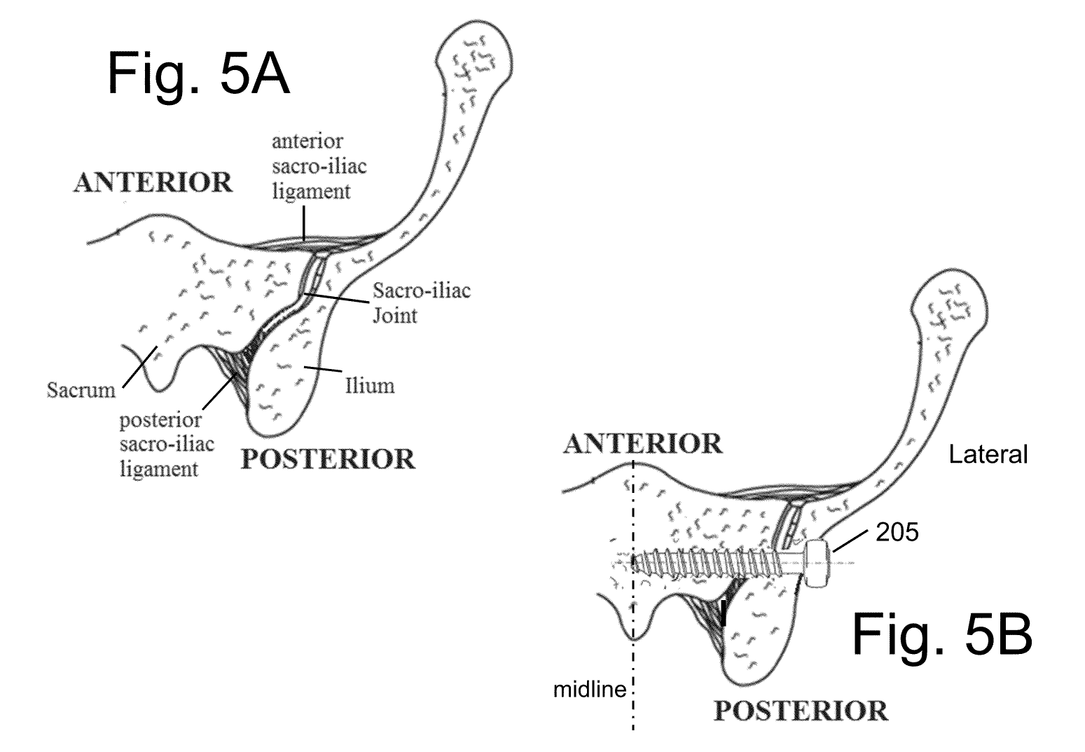

When the painful SI joint is identified, it may be injected with corticosteroids or other medications to reduce pain. If the joint becomes a source of chronic pain, or if the joint and surrounding bones are unstable because of trauma, neoplastic destruction or other disruptive causes, then bone screw fixation of the joint may be performed. FIG. 5A illustrates a diagrammatic cross-sectional view of an SI joint, whereas FIG. 5B illustrates the fixation of the joint with bone screw 205. Those of ordinary skill in the art will appreciate that an actual SI joint may contain features not seen in FIGS. 5A and 5B.

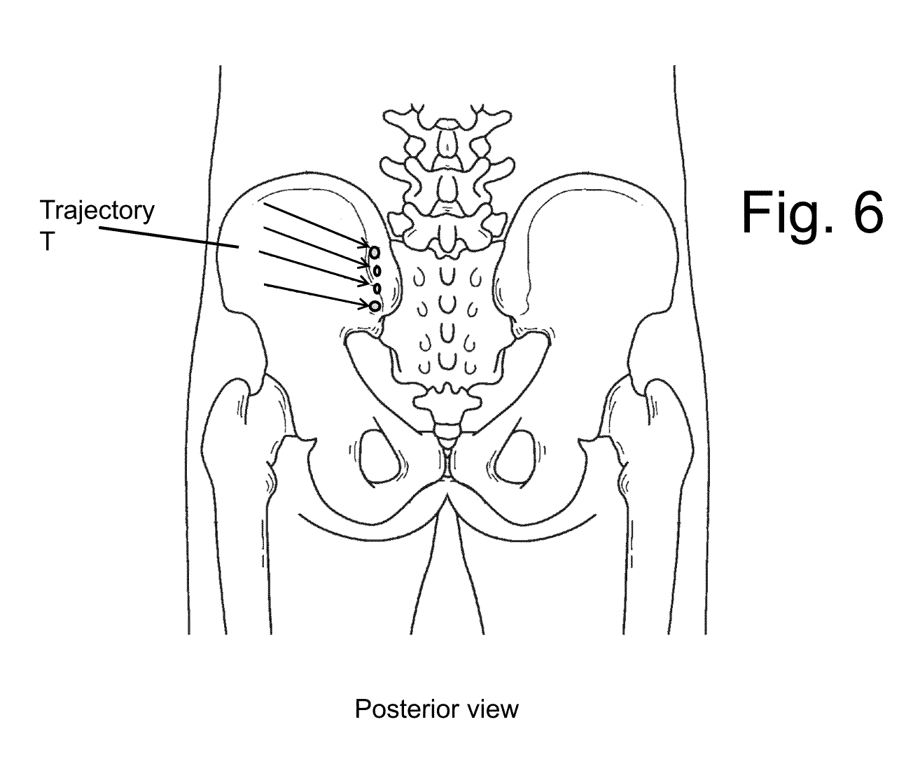

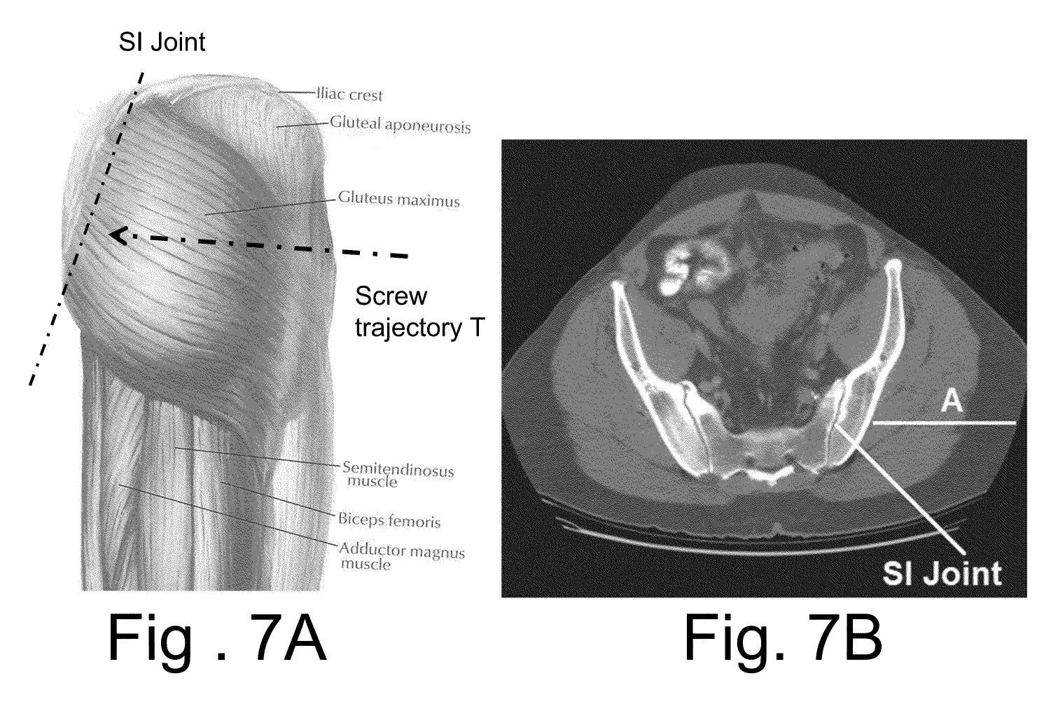

As with provocative injections, fixation screws are positioned substantially within an axial plain and/or substantially perpendicular to the joint. Multiple screws are usually placed across a single joint, as shown by the arrows labeled “Trajectory T” of FIG. 6. Note that according to this approach each screw traverses the large gluteal musculature located posterior and lateral to the target SI joint. FIGS. 7A and 7B show the extent of muscle that must be traversed in order to reach a target SI joint using Trajectory T. In FIG. 7A, a posterior view of the upper aspect of the right lower extremity is diagrammatically shown. The approximate location of a target SI joint and the Trajectory T are shown. While Trajectory T is shown atop the Gluteus Maximus muscle, it is understood that the actual Trajectory T must traverse the gluteal muscle layers. This is illustrated in FIG. 7B, wherein a transverse CT section (i.e., a section parallel to the floor of an upright/standing subject) of an actual subject is shown. Note the extent of distance “At” that must be traversed by a screw following Trajectory T in order to reach the target SI joint.

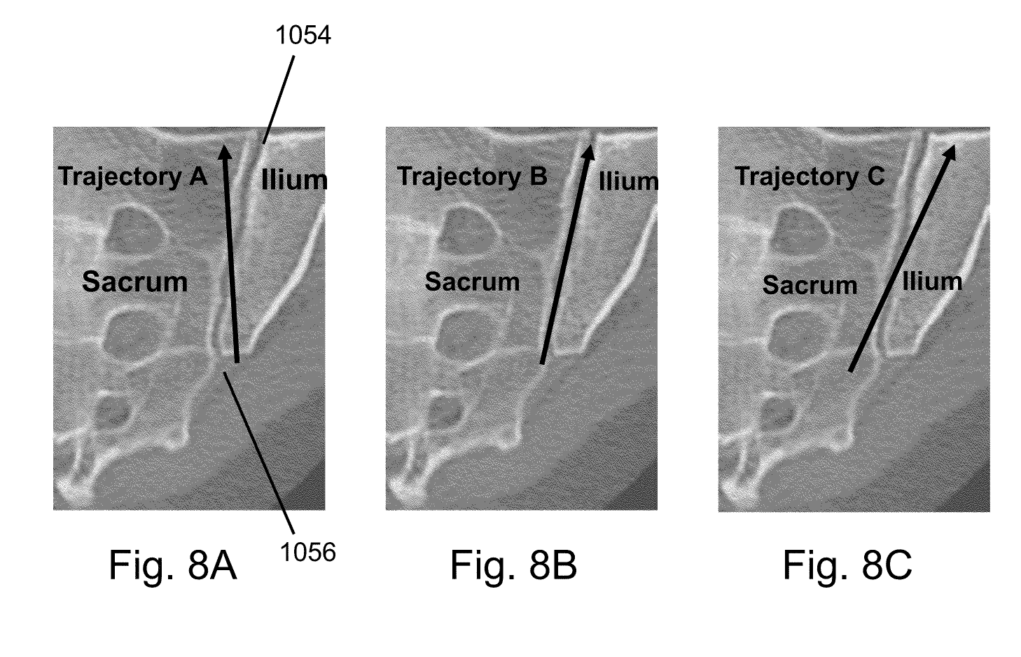

In the present disclosure alternative devices and methods to inject and/or stabilize (including fusion) a target SI joint are disclosed. FIGS. 8A to 8C illustrate Trajectory A to C. To employ any of these trajectories, a subject is placed in the prone (i.e., lying face down) position or onto one side (i.e., substantially positioned in a so-called “lateral decubitus position”). In one exemplary embodiment, the subject's hips are flexed. The inferior aspect/end of the SI joint 1056 is palpated and the level is confirmed by an imaging technique such as, for example, x-rays (including fluoroscopy), CT, MRI, sonography and the like. In most subjects, the inferior aspect the SI joint is superficial and is separated from the skin surface by a modest amount of soft tissues.

While any of the illustrated trajectories A to C (FIGS. 8A to 8C) may be used to access the target SI joint, in one embodiment injection of the joint performed through Trajectory B. In that trajectory, the course of the injection is substantially along the direction of the target joint and extends from 1056 towards 1054. It is further noted that alternative placement of the injection device, such as syringe/needle, from 1054 towards 1056 may be used with equal success. The injectable material is deposited directly from the needle end into the tissue or through an implanted catheter. The catheter can be placed along Trajectory B using any of many methods known in the art for catheter placement. For example, a catheter may be placed over a guide wire that traverses the internal bore of the catheter. Alternatively, the catheter can be passed through the internal bore of a large needle or cannula.

The catheter may be used once and then removed, or it may be left indwelling within the subject for repeat injections over time. It is noted that the catheter may be completely positioned beneath the skin, or alternatively only a segment of it may be beneath the skin while another segment is positioned outside the body. After implantation, the catheter is used to inject the injectable material in a trajectory that is substantially parallel to the course of the target SI joint (as is shown by the arrow of FIG. 8B). When viewed in cross-section, the catheter (or injection needle) is in one embodiment positioned within the posterior sacro-iliac ligament (region 120 of FIG. 3) and adjacent to the target joint. However, at least a partition of the catheter (or injection needle) may also traverse at least a portion of the target SI joint itself.

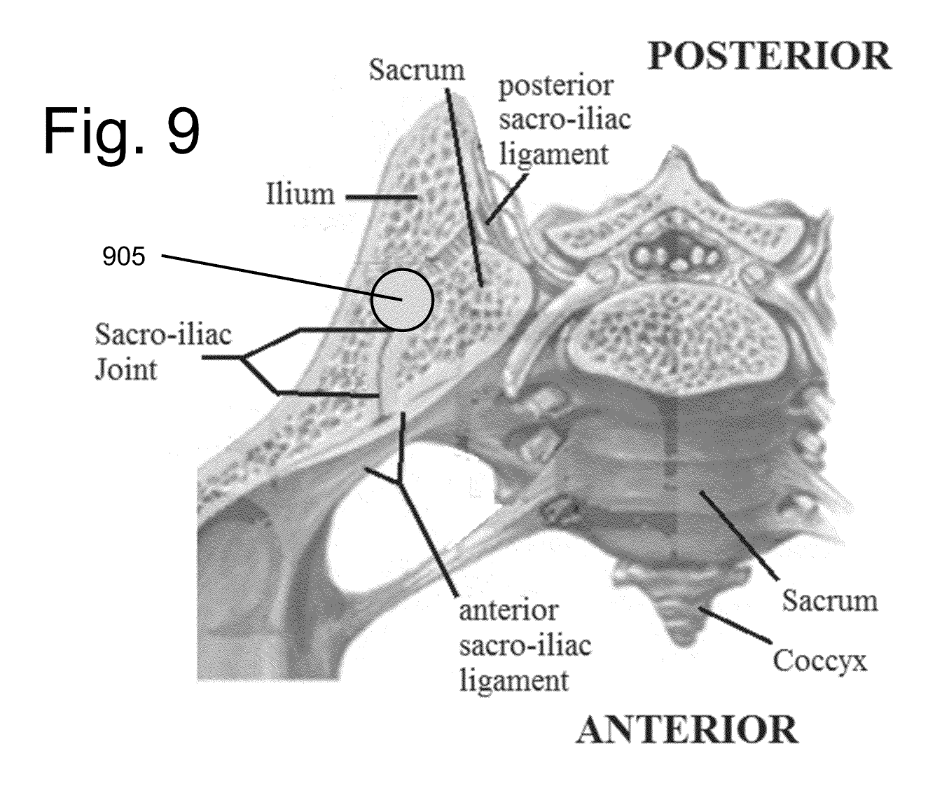

Trajectory B may be also used to implant a bone forming material within and/or adjacent to the target SI joint. For example, a bony defect 905 may be created in the direction of Trajectory B within the posterior sacro-iliac ligament (FIG. 9). The defect may or may not cross the SI joint. The defect is filled with a bone graft (such as, for example, an autograft or allograft bone segment with or without a bone growth promoting factor such as, for example, a Bone Morphogenic Protein) or any bone-forming substance in order to form a bone fusion across the created defect. The defect may be also implanted with a device that is comprised of an outer structural surface and an internal cavity that is configured to accept a bone forming material, the structural surface having openings that permit communication between the bone forming material of the internal cavity and the bone of the ilium and/or sacrum that surrounds the device. In this way, a bone fusion is produced between the ilium and sacrum, the fusion crosses the internal cavity of the device. In one embodiment the devices such as those disclosed in U.S. Pat. Nos. 5,015,247; 5,026,373; 5,192,327; 6,652,584 may be utilized, each of the foregoing being incorporated herein by reference in its entirety.

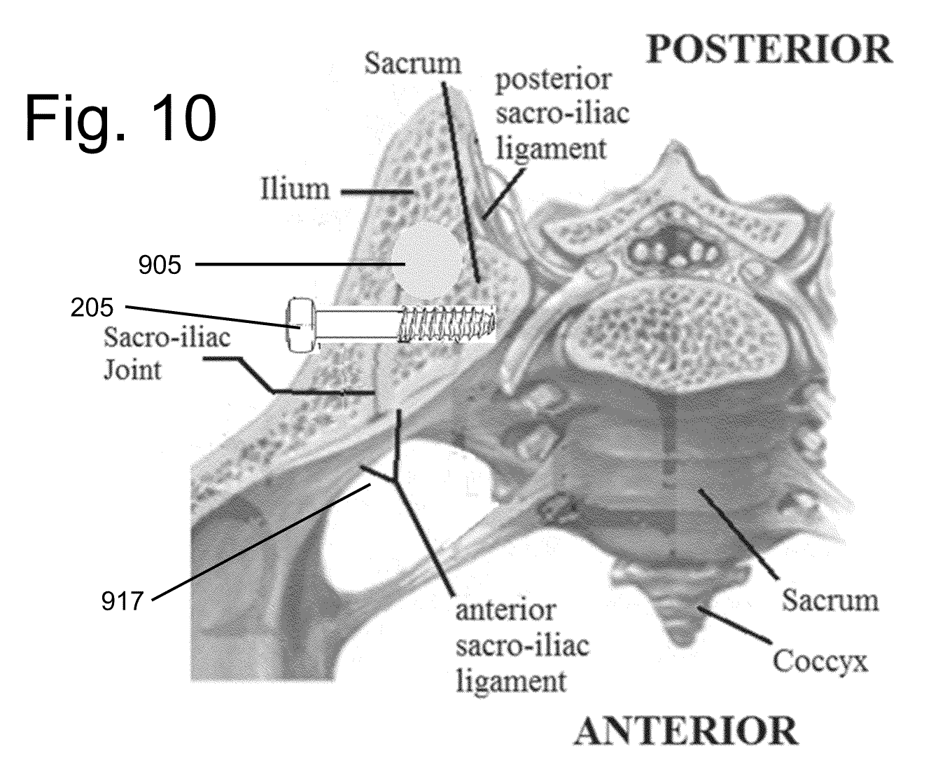

In another embodiment, the surgeon may place his finger anterior to the target SI joint and substantially along a Trajectory B (see 917, FIG. 10) in order to guide the instrumentation. In this maneuver, the finger enters the body of the subject through a skin incision that is in proximity to area 1056.

After placement of bone forming material in or adjacent to the SI joint, it may be also immobilized while the fusion mass forms. The fixation may be performed by placing at least one bone screw 205 through Trajectory T of FIGS. 6 & 7. In one embodiment, two to five bone anchors are placed across a single SI joint and the anchors are positioned in a trajectory substantially perpendicular to the SI joint and extending from a medial to lateral direction. FIG. 10A schematically illustrates a cross section of the SI joint wherein bone forming material (whether alone, or contained within an implant having a structural outer surface and an internal cavity, as described above) is positioned in defect 905. As shown, at least one bone anchor 205 is used to fixate and immobilize the SI joint. Bone anchor(s) 205 may be implanted using any device and/or method desired by the operating surgeon.

In another embodiment, bone anchor(s) 205 are positioned in a percutaneous manner and with concurrent use of an image modality (X-ray, CT, MRI, sonographic imaging and the like). FIGS. 10B-10E illustrate an exemplary drill/screw guide 705 that may be used to position a bone anchor 205 across the SI joint. The device 705 generally comprises a segment 7052 configured to attach onto a member positioned in defect 905, such as a drill used to form the defect 905, a bushing-like member used to transiently occupy defect 905, a bone graft material placed into defect 905 (such as allograft cylindrical bone segment and the like), and/or a fusion device that has been implanted into defect 905 (such as, for example, an implant having a structural outer surface and an internal cavity configured to house a bone graft material—as described above).

As illustrated, the arm 7054 is positioned opposite and substantially parallel to segment 7052. Interconnecting member 7056 connects arm 7054 to segment 7052. Member 7056 maintains segment 7052 in a defined spatial relationship relative to arm 7054 and may be also used by the operator to hold the device 705. Arm 7054 has a distal segment 70542 and a guide segment 715 that is movably attached to the distal segment 70542. Guide segment 715 is configured to translate relative to segment 70542 in the direction K. A threaded set screw 7152 (threads not shown) is used to reversibly immobilize guide segment 715 relative to segment 70542 after the guide segment 715 is placed at its desired position. FIG. 10D illustrates a cross sectional view of guide segment 715. The guide segment 715 has cavity 7154 that is configured to receive complimentary distal segment 70542 of arm 7054, whereas threaded bore 7156 receives threaded set screw 7152. Guide segment 715 is further comprised of tube 725 having internal bore 7252, wherein bore 7252 is configured to guide a bone anchor 205 and to position it in a predetermined spatial orientation relative to defect 905. Note that the bone anchor 205 may be placed anterior to the defect 905 (as shown in FIG. 10A) or posterior to the defect 905 by simply repositioning the guide segment 715 to aim onto a side “A” or a side “B” of longitudinal Axis F of first segment 7052. As illustrated in FIG. 10B, guide segment 715 is shown aiming towards side “B” of Axis F. While not shown, it is appreciated that in another embodiment, the guide may be equally configured to aim and position an anchor 205 substantially on Axis F. Guide segment 715 may be also used as a drill guide, in order to correctly guide a drill that is used to predrill the bore hole prior to actual advancement of anchor 205.

In use, the patient is placed in the prone position. However, it is appreciated that the procedure may be performed with the patient in a lateral decubitus position as well. The notch at the inferior end of the SI joint that is to be fused (1056 of FIG. 2) is palpated where the medially-positioned sacrum meets the laterally-positioned ilium. The position of the inferior end of the SI joint (1056) is confirmed by an imaging modality (such as e.g., X-ray, CT, MRI, sonographic imaging and the like). A small skin incision is made overlying 1056 and a perforating member (such as wire, needle or the like) is preferably passed from 1056 to 1054 substantially along Trajectory B of FIG. 8B. The perforating member may be within the SI joint itself or adjacent to it. If positioned adjacent to the SI joint, the perforating member may be at least partially positioned anterior or posterior to the SI joint. In another embodiment, the perforating member is positioned posterior the SI joint and within the posterior sacroiliac ligament.

A drill is then advanced along the perforating member so as to fowl a bony defect that is partially within the sacrum and partially within the ilium (such as, for example, defect 905 of FIG. 10A). The created defect is implanted with a fusion device having an external shell surface and an internal cavity that contains bone forming material (as described above), or with bone forming material alone.

The SI joint is then immobilized using bone anchors that are placed across the SI joint using Trajectory A (FIG. 8A), Trajectory C (FIG. 8C), a trajectory perpendicular to the SI joint (for example, as shown in FIG. 10A), or any other applicable trajectory for anchor placement. In one embodiment, the anchor is placed using device 705 and advanced via a substantially perpendicular trajectory relative to the SI joint. FIG. 10E shows a schematic illustration of device 705 in use. The patient is shown in the prone position, and the first segment 7052 is positioned in proximity to the inferior end 1056 of the SI joint. The remainder of device 705 is external to the body of the patient. Guide segment 715 is movement in direction K in order to align internal bore 7252 with the desired anchor position in the cephalad-caudad direction of the SI joint. (The SI joint is visualized using an intra-operative imaging modality, or using imaging guidance techniques based on pre-operative imaging of the SI joint.) The guide segment is immobilized relative to distal segment 70542; and internal bore 7252 is used to percutaneously guide the anchor across the SI joint. In another variant, the anchor insertion site may be pre-drilled using a drill that is also guided by bore 7252.

More than one anchor may be placed across the SI joint using device 705. This is performed by translating guide segment 715 cephalad/caudad along direction K and using bore 7252 to substantially position multiple anchors in a configuration similar to that of FIG. 6.

Using this method, bone forming material is placed at least partially within the target SI joint (and/or at least partially within a segment of the posterior sacro-iliac ligament positioned posterior to the target SI joint). One or more bone fixation anchors are then placed across the target SI joint (and/or across a segment of the posterior sacro-iliac ligament positioned posterior to the target SI joint) in order to immobilize the target SI joint. The bone fixation anchor(s) are, in one variant, positioned to form an angle of 65 to 115 degrees relative to the longitudinal axis of the target SI joint. At least one bone fixation anchor is positioned substantially perpendicular to the longitudinal axis of the target SI joint in another variant.

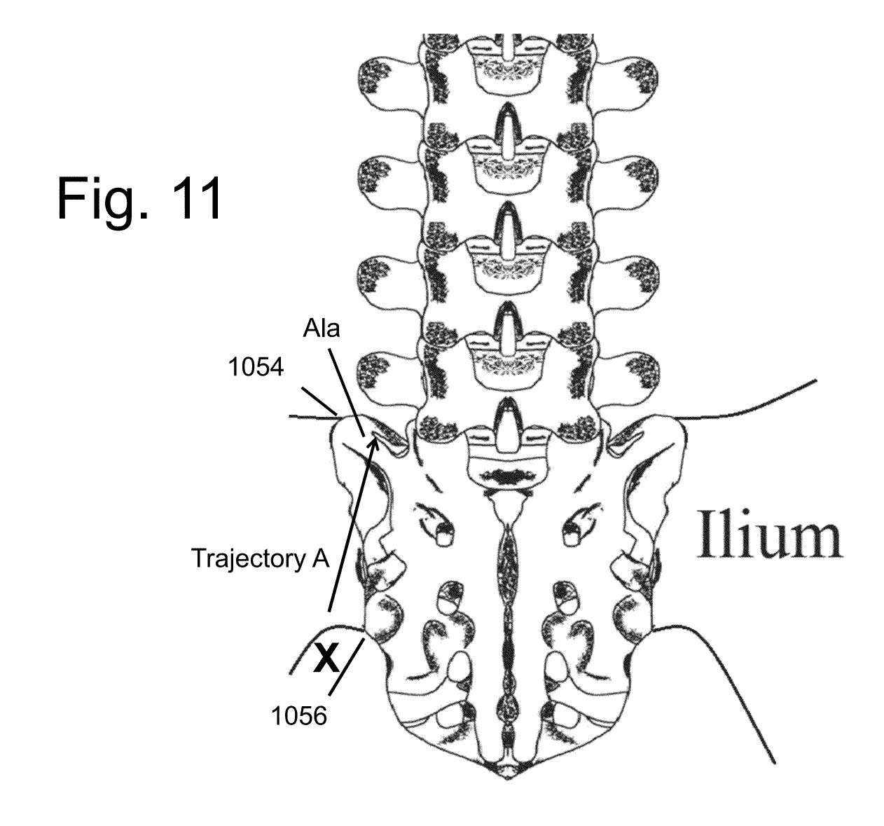

As noted, a fixation member (which may be a bone screw or other anchor) may be positioned across the SI joint using Trajectory A and/or Trajectory C. The fixation device may cross the actual SI joint space or be positioned entirely within the posterior Sacro-iliac ligament. FIG. 11 illustrates use of Trajectory A, wherein the fixation member enters the ilium substantially at point “X” (and in proximity to 1056) and is directed towards the sacral ala. Point “X” is adjacent to the inferior aspect of target SI joint and is, in one embodiment, on the posterior bony surface of the ilium or its inferior bony surface. Trajectory A forms an acute angle with the longitudinal axis of the target SI joint. As show in FIG. 11, angle A donates an angle formed by the Trajectory A and the longitudinal axis of the target SI joint. In one embodiment, angle A is less than 45 degrees. In another embodiment, angle A is less than 30 degrees. In yet another embodiment, angle A is less than 15 degrees. A fixation member placed through Trajectory A extends substantially along the cephalad-caudad direction of the target sacro-iliac joint, entering the ipsilateral ilium bone immediately lateral to the sacro-iliac joint, extending medially across the sacro-iliac joint or a posterior sacro-iliac ligament positioned posterior to the target sacro-iliac joint, and then extending into the lateral bony aspect of the sacrum

While Trajectory A is shown and described as extending cephalad from an inferior ilium bone entry site, the course of Trajectory A may be advanced caudad from a proximal cephalad sacral bone entry point to distal caudad ilium bone site in another embodiment.

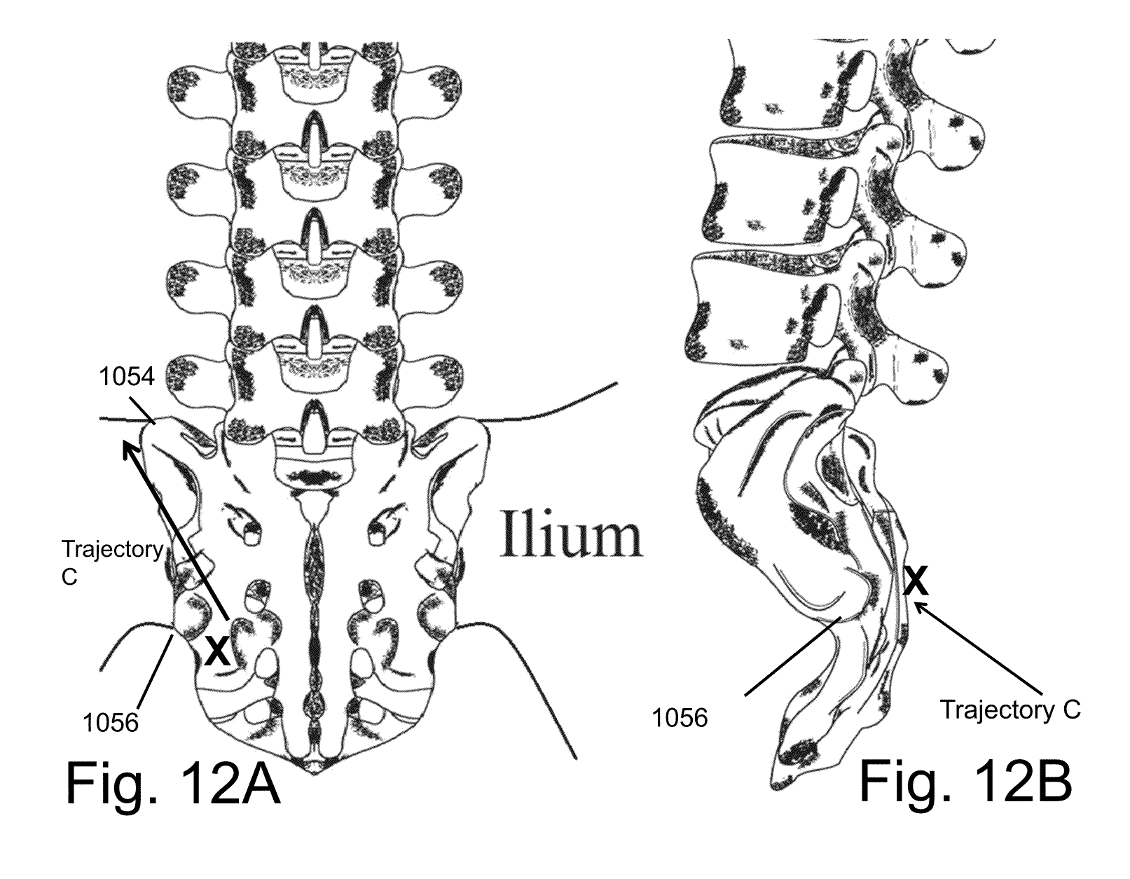

FIGS. 12A (posterior view) and 12B (lateral view) illustrate use of Trajectory C, wherein the fixation member enters the posterior aspect of the Sacrum substantially at point “X” (and in proximity to 1056) and is directed at the superior aspect of the ilium. Point “X” is adjacent to the inferior aspect of target SI joint and is, in one embodiment, located on the posterior bony surface of the sacrum. Trajectory C forms an acute angle with the longitudinal axis of the target SI joint (as is illustrated in FIG. 11 for Trajectory A). That is, an angle formed by Trajectory C and the longitudinal axis of the target SI joint is less than 90 degrees. In one embodiment, the angle is less than 45 degrees. In another embodiment, the angle is less than 30 degrees. In yet another embodiment, the angle is less than 15 degrees.

A fixation member is placed through Trajectory C and extends substantially along the cephalad-caudad direction of the target sacro-iliac joint, entering a posterior surface of the sacrum bone medial to the target sacro-iliac joint, extending laterally across the target sacro-iliac joint (or the posterior sacro-iliac ligament positioned posterior to the target sacro-iliac joint), and extending into a medial aspect of the ilium. While Trajectory C is shown and described as extending cephalad from an inferior sacrum bone entry site, it is equally contemplated that the course of Trajectory C could be advanced caudad from a proximal cephalad ilium bone entry point to distal caudad sacrum bone site. Finally, the fixation member may be positioned using Trajectory A, B or C, even when no bone graft material has been placed across the SI joint.

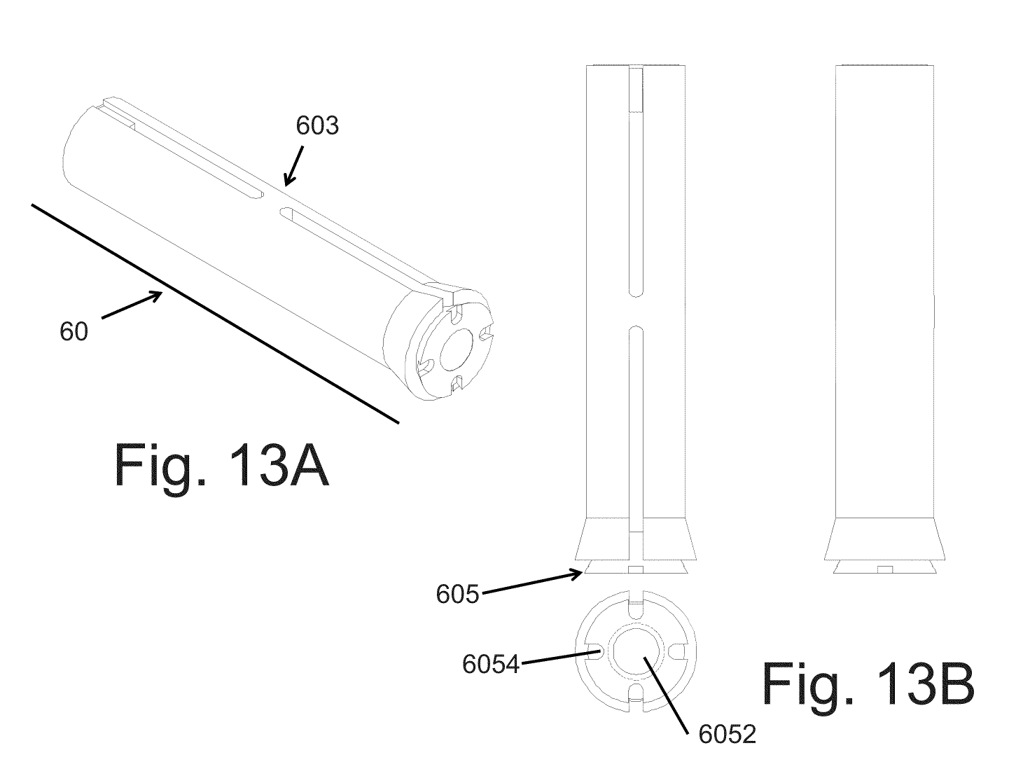

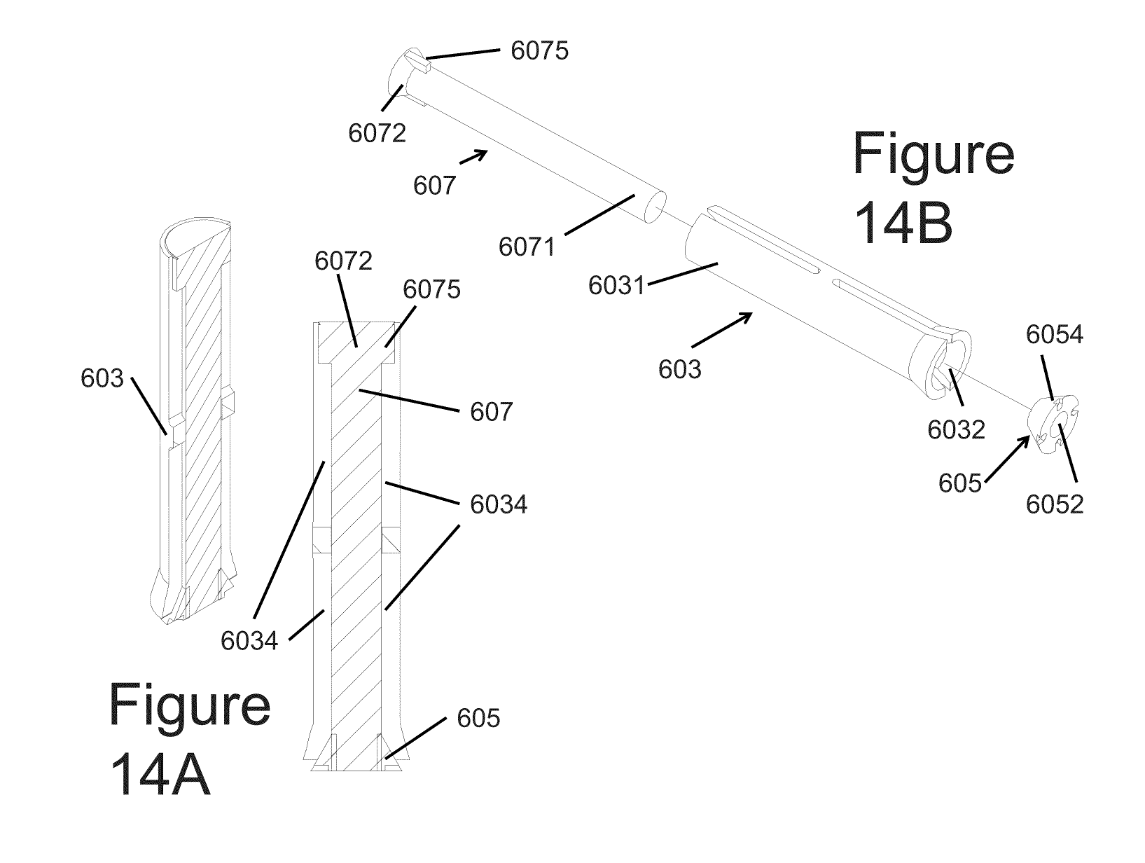

FIGS. 13 and 14 illustrate an exemplary fixation member that may be used with Trajectories A-C of FIGS. 8A-8C. (Note that a conventional bone screw may serve as an alternative fixation member that uses Trajectories A-C.) Fixation member 60, in one embodiment comprises an external member 603 and internal members 605 and 607, wherein members 605 and 607 threadedly engage one another. Elongated member 603 has an outer surface 6031 and an internal bore 6032. At least one expansion joint 6034 extends from the outer surface 6031 to the internal bore 6032. The outer surface 6031 may be smooth or textured. It may contain at least one protrusion and/or at least one indentation that enhance fixation of member 603 into the surrounding bone. The outer surface may be at least partially threaded. Further, member 603 may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant. Finally, member 603 may be at least partially made of a bone.

Member 607 has a first threaded end 6071 (threads not shown) and head 6072 at an opposing end. Protrusions 6075 are adapted to engage an indentation of member 603 (such as, for example, expansion joint 6034) and prevent the rotation of member 607 relative to member 603. Member 605 contains a threaded internal bore 6052 (threads not shown) that cooperatively engage the threads of end 6071 of member 607. Member 605 further contains at least one indentation and/or protrusion 6054 that is adapted to accept a complimentary segment of a screw driver or other device for engaging the member 605.

In application, an SI joint is targeted for fixation (and/or fusion) and the target SI joint is radiographically identified. A bore hole is drilled along Trajectory A or Trajectory C, wherein the bore hole traverses the ilium and the sacrum on the same side of the midline as the target SI joint. The bore hole may at least partially traverse the target SI joint or it may rest completely outside of the target SI joint. Fixation member 60 is driven into the creased bore hole. Member 605 is threadedly advanced relative to member 607 so that the distance form member 605 to head 6075 is decreased. This action forces the expansion of member 603 along expansion joints 6034. In a preferred embodiment, a first end segment of member 60 is positioned at least partially within the ilium, while a second end segment of member 60 is positioned at least partially within the Sacrum. In this way, the expanded member 60 can function to fixate the ilium to the Sacrum on the side of the target SI joint. In one embodiment, bone graft material may be concurrently positioned along Trajectory B, as described above. This method would provide bone fixation as well as bone graft material in order to immobilize and fuse the target SI joint.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials, and the like. Any components may be also coated/made with osteo-conductive bio-active materials (such as e.g., deminerized bone matrix, hydroxyapatite, and the like) and/or osteo-inductive bio-active materials (such as e.g., Transforming Growth Factor “TGF-B,” Platelet-Derived Growth Factor “PDGF,” Bone-Morphogenic Protein “BMP,” and the like) that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, the system or any of its components can also be entirely or partially made of a shape memory material or other deformable material.

The shown embodiments are illustrative and do not limit the scope of the invention. At a minimum, additional embodiments of the present invention can be created by one of ordinary skill using various combinations of the embodiments illustrated herein.

What is claimed is:

1. A method for performing a therapeutic or diagnostic procedure on a target sacro-iliac joint positioned between an adjacent sacrum bone and ilium bone, comprising:
accessing a region in proximity to an inferior end of said target sacro-iliac joint;
forming a corridor extending from said accessed region to a superior end of said target sacro-iliac joint, said corridor being substantially aligned along a longitudinal axis of said target sacro-iliac joint; and
performing said therapeutic or diagnostic procedure at least partially through said formed corridor.

2. The method of claim 1, wherein said therapeutic or diagnostic procedure comprises injecting medication within said target sacro-iliac joint.

3. The method of claim 1, further comprising implanting a catheter at least partially within said formed corridor, said catheter enabling repeat performance of said therapeutic or diagnostic procedure.

4. The method of claim 2, further comprising utilizing said injection to diagnose a source of pain.

5. The method of claim 1, further comprising creating cavity at least partially along said formed corridor, said cavity spanning at least one of: (i) a segment of said target sacro-iliac joint, or (ii) a segment of a posterior sacro-iliac ligament positioned posterior to said target sacro-iliac joint.

6. The method of claim 5, further comprising positioning a bone forming material within said created cavity, said bone forming material forming a bone fusion mass between said sacrum and ilium bone.

7. The method of claim 6, further comprising utilizing at least one bone fixation anchor to substantially immobilize said sacrum bone relative to said ilium bone.

8. The method of claim 6, further comprising positioning a fusion implant within said created cavity, said fusion implant comprising an outer surface member and an internal cavity configured to house at least a segment of said bone forming material.

9. The method of claim 7, further comprising attaching a guide device to at least a portion of said created cavity, said guide device being configured to enable advancement of said bone fixation anchor.

10. A method for placing an implant within a target sacro-iliac joint of an adjacent sacrum bone and ilium bone, comprising:
accessing an inferior segment of said target sacro-iliac joint;
forming a corridor from said accessed position towards an ipsilateral portion of said sacrum bone, said corridor extending substantially along a cephalad-caudad direction of said target sacro-iliac joint, entering said ilium bone immediately lateral to said sacro-iliac joint, extending medially across said sacro-iliac joint or a posterior sacro-iliac ligament positioned posterior to said sacro-iliac joint, and extending into a lateral aspect of said sacrum bone; and
advancing said implant into said formed corridor, said implant when fully advanced being positioned such that a distal end thereof is cephalad to a proximal end thereof.

11. The method of claim 10, wherein act of advancing said implant into said formed corridor comprises advancing a bone anchor therein.

12. The method of claim 10, wherein said act of forming a corridor comprises forming an angle of less than 45 degrees between a longitudinal axis of said formed corridor and a longitudinal axis of said target SI joint.

13. The method claim 10, wherein said implant is at least partially manufactured of a metallic alloy.

14. The method of claim 10, wherein said implant is at least partially manufactured of a plastic material.

15. The method of claim 10, further comprising facilitating bone formation with said adjacent sacrum bone via a bioactive substance contained in said implant.

16. A method of immobilizing of a target sacro-iliac joint positioned between an adjacent sacrum bone and ilium bone, comprising:
accessing a region in proximity to an inferior segment of said target sacro-iliac joint;
forming a corridor from said accessed region towards an ipsilateral portion of said ilium bone, said corridor extending in a cephalad-caudad direction and substantially along a direction of said target sacro-iliac joint, said corridor entering a posterior surface of said sacrum bone medial to said target sacro-iliac joint, extending laterally across said sacro-iliac joint or a posterior sacro-iliac ligament positioned posterior to said sacro-iliac joint, and entering a medial aspect of said ilium bone; and
advancing a bone anchor at least partially along said formed corridor;
wherein, when fully advanced, said bone anchor being positioned with a distal end in proximity to a cephalad end of said target sacro-iliac joint.

17. The method of claim 16, wherein an angle formed by a longitudinal axis of said formed corridor and a longitudinal axis of said target SI joint is less than 45 degrees.

18. The method of claim 16, wherein said bone anchor is at least partially manufactured from a metallic alloy.

19. The method of claim 16, wherein said bone anchor is at least partially manufactured from a plastic material.

20. The method of claim 16, further comprising facilitating bone formation with said adjacent sacrum bone via a bioactive substance contained in said implant.

21. A method of substantially immobilizing a target sacro-iliac joint positioned between an adjacent sacrum bone and ilium bone, comprising:
accessing a region in proximity to an inferior segment of said target sacro-iliac joint;
forming a corridor from said accessed inferior segment and towards a superior end of said target sacro-iliac joint, said corridor being substantially aligned to extend along a cephalad-caudad direction of a longitudinal axis of said target sacro-iliac joint;
creating a cavity spanning at least one of a segment of said target sacro-iliac joint, or a segment of a posterior sacro-iliac ligament positioned posterior to said target sacro-iliac joint, said cavity extending at least partially along said formed corridor; and
positioning a bone forming material within said created cavity, said bone forming material being configured to form a bone fusion mass between said adjacent sacrum and said ilium bone.

22. The method of claim 21, further comprising positioning an allograft bone segment within said created cavity.

23. The method of claim 21, further comprising positioning a fusion device within said created cavity, said fusion device comprising an outer structural surface and an internal cavity configured to accept a bone forming material, said structural surface comprising plurality of openings configured to permit communication between said bone forming material of said internal cavity and at least one of said sacrum bone and said ilium bone.

24. The method of claim 21, further comprising advancing at least one bone fixation anchor at least partially between said adjacent sacrum and said ilium bone and across the target sacro-iliac joint or a segment of a posterior sacro-iliac ligament positioned posterior to the target sacro-iliac joint.

25. The method of claim 24, wherein said act of advancing comprises positioning said at least one bone fixation anchor such that a distal end thereof is in proximity to a cephalad end of said target sacro-iliac joint.

26. The method of claim 25, wherein said act of forming a corridor comprises a longitudinal axis of said at least one bone fixation anchor and said longitudinal axis of said target SI joint form an angle of less than 45 degrees.

27. The method of claim 24, wherein said act of advancing comprises positioning said at least one bone fixation anchor such that a distal end thereof is cephalad to a proximal end thereof.

28. The method of claim 27, wherein an angle formed by the longitudinal axis of said at least one bone fixation anchor and a longitudinal axis of said target SI joint is less than 45 degrees.

29. The method of claim 24, wherein an angle formed by a longitudinal axis of said at least one bone fixation anchor and a longitudinal axis of said target SI joint is less than 65 degrees.

30. The method of claim 24, wherein an angle formed by a longitudinal axis of said at least one bone fixation anchor and a longitudinal axis of said target SI joint is less than 115 degrees.

31. The method of claim 24, wherein said act of advancing said at least one bone fixation anchor comprises percutaneous advancement thereof.

32. The method of claim 24, wherein said act of advancing said at least one bone fixation anchor comprises advancement via a guide instrument having a first segment attached to said created cavity and second segment configured to guide said at least one bone fixation anchor.

33. The method of claim 32, further comprising using said guide instrument to position said at least one bone fixation anchor such that a longitudinal axis thereof is substantially perpendicular to said longitudinal axis of said target sacro-iliac joint.
